(19) United States
(12) Patent Application Publication
(10) Pub. No.: US 2005/0015057 A1
(43) Pub. Date: Jan. 20, 2005

(54) Cylindrical piston press foot

(76) Inventors: Soren Steenfeldt-Jensen, Hornbaek (DK); Peter Nordland Jensen, Kastrup (DK)

Correspondence Address: NOVO NORDISK, INC.;PATENT DEPARTMENT, 100 COLLEGE ROAD WEST, PRINCETON, NJ, 08540, US

(21) Appl. No.: 10/720,903

(22) Filed: Nov. 24, 2003

(30) Foreign Application Priority Data
Nov. 28, 2002 (DK) PA 2002 01836

(51) Int. Cl. A61M 5/315

(52) U.S. Cl. 604/228

(57) ABSTRACT

Disclosed is a cylindrical piston press foot mounted on a coupling element having a part with an enlarged diameter connected through a part with reduced diameter to the end of a piston rod. The device is useful in injection devices, mespecially devices by which a liquid medication is apportioned in individual doses from a cartridge.

### CROSS-REFERENCE TO RELATED APPLICATIONS

This application claims priority under 35 U.S.C. 119 of Danish application no. PA 2002 01836 filed Nov. 28, 2002, the contents of which are fully incorporated herein by reference.

The invention relates to injection devices and especially to devices by which a liquid medicine is apportioned in individually set doses from a cartridge comprising a vessel having a cylindrical wall, and a first and a second end, where the fist end is closed a membrane and the second end is closed by a piston which can by a piston rod be pressed into the cartridge a distance which is proportional with a set dose, whereby liquid stored in the space defined by the membrane, the piston, and the cylindrical wall can be pressed out through an injection needle which pierces the membrane.

More specifically the invention relates to a cylindrical piston press foot mounted on a coupling element having a part with enlarged diameter connected through a part with reduced diameter to the end of a piston rod,

The piston rod forms a part of a dose setting and injecting mechanism by which a dose can be set by rotating a button and subsequently be injected by pressing this button whereby the piston rod is moved outward from the dose setting and injection mechanism and presses the piston of a cartridge successively into the cartridge.

The pressure delivered by the piston rod to the piston is distributed over the entire end surface of the piston or over a chosen area of this surface, which distribution is obtained by a press foot attached to the end of the piston rod adjacent to the piston to be inserted between said piston rod and said piston.

A mainly used way of attachment of a press foot to a piston rod is to provide the end of the piston rod with a part with enlarged diameter, e. g. a sphere or a sphere like curved protrusion which is adopted in a mating space provided in the pressure foot at the centre thereof, where an opening giving access to said space has a diameter which is smaller than the maximal diameter of said curved protrusion, the diameters being seen perpendicularly to the axis of the piston rod. The press foot may be mounted to the piston rod by clicking the said protrusion through said access opening into said space. To enable this the diameter of the access opening must be large and deformable enough to allow said protrusion to enter the space through said access opening when the piston rod with a moderate pressure is pressed against the pressure foot.

In durable devices which are used for administration of the content of more cartridges wherein one cartridge when empty is replaced by a new full one, the piston rod is moved outward to protrude from the dose setting and injection part a distance corresponding to the length of cartridge. Before a new cartridge can be mounted the piston rod must be returned into the dose setting and injection unit. This is obtained by either screwing or pressing the piston rod back piston rod back into the dose setting and injection part. When the piston rod is pressed with by a finger the pressure may often deviate from a pure axial pressure whereby the pressure foot on which the pressure is exerted may capsize so that the protrusion on the piston rod clicks out of the cavity in the press foot and the press foot falls of. In such case it may difficult to the user to click the pressure foot onto the piston rod again and if he succeeds the press foot may be only partly clicked on so that the piston rod can be axially moved a short distance before the pressure foot moves the piston. If the cavity shall be given a shape so that the access opening to the cavity does not allow such click on or click off the press foot must either be moulded around the protrusion of the piston rod or it must at least be heated and deformed after the mounting.

It is an objective of the invention to provide a cylindrical piston press foot which can be mounted rotatably on a protrusion at the end of a piston rod in a way which preclude clicking off of the pressure foot when a non axial pressure is transmitted between said pressure foot and the piston rod.

This is obtained by a cylindrical piston press foot mounted on a mainly spherical coupling element at the end of a piston rod, which press foot is characterised in that it comprises two identical half cylindrical halves of which one is rotated 180° relative to the other about the piston rod axis so that the two halves can be moved toward each other to fit each other along mating surfaces along a diameter of the resulting press foot, the mating surfaces being provided with cavities which together form a central cavity in the resulting press foot when the mating surfaces are brought into abutment with each other, which central cavity accommodates the coupling element at the end of the piston rod so that a joint between the piston rod and the press foot is established.

The two halves may be welded together over the mating surfaces.

Alternatively each of the two halves of the press foot may at its surface mating a corresponding surface of an opposite half be provided with a set of mating snap lock devices positioned symmetrical about its cavity so that the set of snap lock devices on one half can snap together with the set of snap lock devices of the other half when the mating surfaces of the two halves are moved into abutment with each other.

The mating snap lock devices may consist of a bore and a tongue fitting into said bore, respectively.

The bore may pass all the way through the press foot and the end of the tongue must be shaped to flush with the cylindrical wall of the press foot, when the two halves are joined together.

At the root of the tongue recesses may be formed on each side of the press foot which recesses runs in the longitudinal direction of the tongue and the bottom of which except for a deeper depression at an inner end is flush with the tongue, the snap lock element mating the tongue comprising flexible arms shaped and placed so at each side of the bore that they will fill out the said recesses when two elements are moved towards each other with the respective tongues received in the respective bores to form a press foot.

The arms are at their free ends provided with hooks which engage the depressions at the inner ends of the recesses.

In the following the invention is described in further details with references to the drawing, wherein

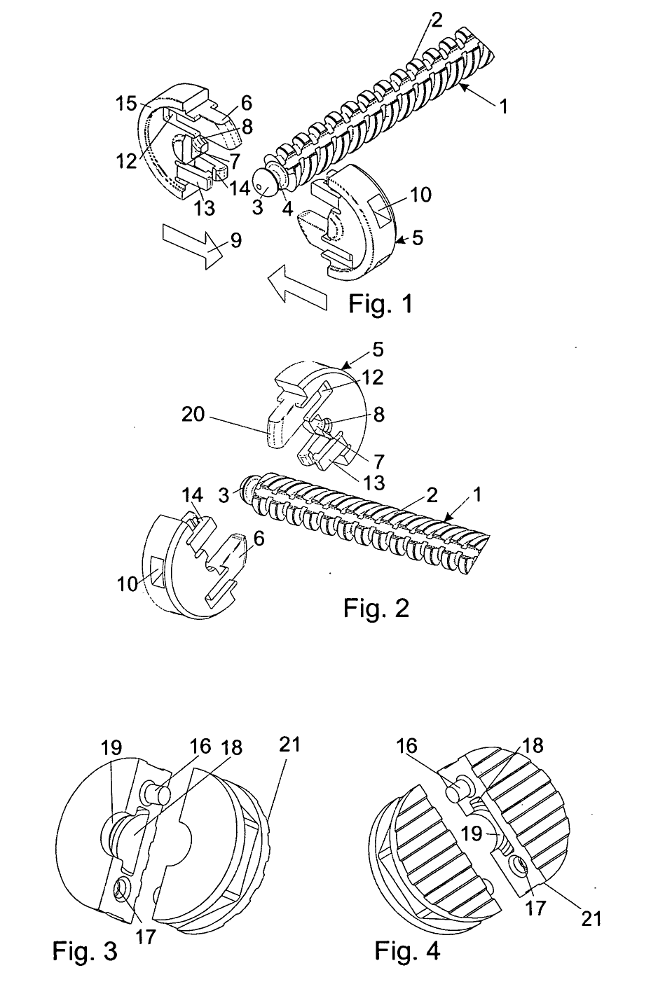

FIG. 1 shows a perspective view of a spindle shaped piston rod and a pair of press foot halves with snap locks ready for mounting onto an end of the spindle,

FIG. 2 shows a perspective view of the spindle and the press foot halves seen in FIG. 1 but form another angle,

FIG. 3 shows another embodiment of a pair of press foot halves ready for being mounted onto an end of a spindle

FIG. 4 shows the press foot halves in FIG. 3 seen from another angle.

In FIG. 1 a spindle 1 which forms a piston rod in a not shown injection device is provided with a thread 2 which cooperates with the dose setting device of the not shown pen. At a free end of the piston rod which project from the dose setting device is provided with a spherical member 3 which is by a neck 4 connected to the free end of the piston rod.

A pair of congruent piston halves 5 are shown opposite each other so that on the surfaces which are going to be brought into abutment with each other a tongue 6 on one half lies opposite a bore 1 0 in the other half and visa versa. By arrows 9 it is indicated how it is intended to move the press foot halves towards each other so that cavities 7 in each press foot half are juxtaposed to form a resulting cavity which accommodates the spherical projection 3 of the piston rod. When the two press foot halves are abutting each other along their mating surfaces a short cylindrical wall 8 forms an opening to the cavity 7 in which opening the neck 4 of the projection 3 at the end of the spindle 1 is accommodated, the diameter of the opening being so small that the spherical protrusion 3 cannot be drawn through it without damaging either the spindle projection or the press foot.

At each side of a press foot half a recess 11 in the direction of the tongue 6 is provided, said recess having at an inner end a depression 12. The same way each press foot half has at each side a resilient snap lock arm 13 adjacent to its bore 10 and projecting from the side forming a diameter in the resulting press foot, which snap lock arm is shaped to be accommodated in the recess 11 of another press foot half when two press foot halves are moved towards each other to form a press foot. At their free ends the snap lock arms have hooks 14 which grip into the depressions 12 at the inner ends of the recesses 11 in aopposite press foot half.

The shown press foot halves are so designed that the resulting press foot has an annular ridge 15 along its perimeter on the side abutting a piston, when the press foot is mounted in a syringe. By designing the pres foot halves so that an opening 8 accommodating the neck 4 of the spindle 1 is only provided at the side of the press foot turning away from the piston, the risk for erroneous mounting of the press foot is eliminated.

FIG. 3 and 4 show another embodiment of a piston press foot which is designed to be assembled by welding of the two halves instead of snapping them together.

Again a pair of congruent piston halves 5 is shown opposite each other. The surfaces 15 which are going to be brought into abutment with each other are plane but are provided with a pin 16 and a bore 17 so positioned that the pin of one half fits into the bore of the other half when the halved are moved towards each other. The established pin/bore connections keep the halves in the correct position for being welded to each other all over their abutting plane surfaces. An ultra sonic welding can be used. The cavity 18 in this embodiment is designed to accommodate a disc shaped coupling element mounted at the end of a piston rod on a neck part which extends through a central opening in the press foot, which opening is formed by cylindrical walls 19 adjacent to the cavity 18.

1. A cylindrical piston press foot mounted on a coupling element having a part with enlarged diameter connected through a part with reduced diameter to the end of a piston rod, wherein the foot comprises two identical half cylindrical halves of which one is rotated 180° relative to the other about the piston rod

2. A cylindrical piston press foot according to claim 1, wherein each half is so designed that the two halves can be moved toward each other to fit each other along mating surfaces along a diameter of the resulting press foot, the mating surfaces being provided with cavities which together form a central cavity in the resulting press foot when the mating surfaces are brought into abutment with each other, which central cavity accommodates the part with enlarged diameter at the end of the piston rod so that a joint between the piston rod and the press foot is established.

3. A cylindrical piston press foot according to claim 2 wherein the two halves are welded together over the mating surfaces.

4. A cylindrical piston press foot according to claim 2 wherein each of the two halves of the press foot at its surface mating an opposite half is provided with mating snap lock devices symmetrical about its cavity so that the snap lock device on one half can snap together with a mating snap lock devices of the other half when the mating surfaces of the two halves are moved into abutment with each other.

5. A cylindrical piston press foot according to claim 4 wherein the mating snap lock devices consists of a bore and a tongue fitting into said bore, respectively.

6. A cylindrical piston press foot according to claim 5 wherein the bore passes all the way through the press foot and the end of the tongue is shaped to flush with the cylindrical wall of the press foot, when the two halves are joined together.

7. A cylindrical piston press foot according to 5, wherein the interconnection with the tongue recesses each having a bottom are formed on each side of the press foot, which recesses runs in the longitudinal direction of the tongue, and that the bottom of the recesses except for a deeper depression at an inner end is flush with the tongue, the snap lock element mating the tongue comprising flexible arms shaped and placed so at each side of the bore that they will fill out the said recesses when two halves are moved towards each other with the respective tongues received in the respective bores to form a press foot.

8. A cylindrical piston press foot according to claim 7, characterised in that he arms have free ends provided with hooks which engage the depressions at the inner ends of the recesses at the root of the tongues.
